(19) United States
(12) Patent Application Publication
(10) Pub. No.: US 2025/0114784 A1
(43) Pub. Date: Apr. 10, 2025

(54) URINE TESTING KIT

(71) Applicants: YELLOSIS, INC., Seoul (KR); SEOUL NATIONAL UNIVERSITY R&DB FOUNDATION, Seoul (KR)

(72) Inventors: Yu Kyung TAK, Seoul (KR); Jong Gun LEE, Gyeonggi-do (KR); Noo Li JEON, Seoul (KR); Keunhwan PARK, Seoul (KR); Jung Seub LEE, Seoul (KR)

(21) Appl. No.: 18/294,617

(22) PCT Filed: Jul. 12, 2022
(86) PCT No.: PCT/KR2022/010092

§ 371 Date: Feb. 2, 2024

(30) Foreign Application Priority Data
Aug. 11, 2021 (KR) 10-2021-0106115

(51) Int. Cl. B01L 3/00

(52) U.S. Cl. CPC B01L 3/5023; B01L 2300/069; B01L 2300/0825; B01L 2300/087

(57) ABSTRACT

The present invention relates to a urine testing kit. The objective of the present invention is to provide a urine testing kit in which the effects of humidity, non-uniformity of reagent reactions, and interference between items are minimized, and fluid can easily flow into the kit, thus increasing the reaction efficiency with urine.

### TECHNICAL FIELD

The present application claims the benefit of priority based on Korean Patent Application No. 10-2021-0106115 filed on Aug. 11, 2021, the entire disclosure of which is incorporated as a part of this specification.

The present disclosure relates to a urine testing kit, and more particularly to a urine testing kit which minimizes an effect of humidity, reagent reaction non-uniformity, and an effect of interference between items, and facilitates fluid inflow, thereby increasing efficiency of reaction with urine.

### BACKGROUND ART

In testing of urine, creating a moisture-proof environment when storing urine test strips, uniformly distributing urine on urine strips when urinating, and preventing various test strips for multiple test items from interfering with each other are important factors for improving accuracy of urine testing.

In order to prevent urine test strips from being exposed to moisture, a solution to moisture-proof efficiency is required, but in general, all that is done for moisture proofing of urine test strips is to protect the test strips from surrounding humidity by a desiccant enclosed in a packaging case along with the urine test strips.

Further, existing urine test strips have problems in that low discoloration uniformity within test reagent paper and test accuracy is reduced due to color change interference with other test items.

Therefore, there is a need for urine test strips with improved accuracy by solving the above problems.

In addition, if urine test strips with tested urine contaminants are stored for a long time, unsanitary problems such as bad odor and contamination may occur, so urine test strips formed of a material that can be disposed of in sewage immediately after use are necessary.

### DISCLOSURE OF THE INVENTION

### Technical Goals

The present disclosure relates to a urine testing kit, and an object of the present disclosure is to provide a urine testing kit which minimizes an effect of humidity, reagent reaction non-uniformity, and an effect of interference between items, and facilitates fluid inflow, thereby increasing efficiency of reaction with urine.

Technical objects to be achieved by the present disclosure are not limited to the technical objects mentioned above, and other technical objects not mentioned may be clearly understood by those skilled in the art to which the present disclosure pertains from the following description.

### Technical Solutions

A urine testing kit of the present disclosure includes a reagent paper which reacts with urine; a substrate part having a seating groove formed on its upper surface in which the reagent paper is seated; and a cover part which is coupled to the upper surface of the substrate part and covers an entrance of the seating groove, wherein a pore hole may be formed in the cover part through which the urine sprayed on an upper surface of the cover part is injected into the seating groove.

### Advantageous Effects

A urine testing kit of the present disclosure includes a cover part formed by a stomata simulated cover structure, so that it is possible to minimize the effect of humidity on the reagent paper and increase the uniformity of reagent paper reaction.

In the urine testing kit of the present disclosure, each of a plurality of reagent papers is located in a space divided from each other, thereby allowing interference in results between the plurality of reagent papers to be eliminated.

In the urine testing kit of the present disclosure, a material of the cover part may be a hydrophilic and biodegradable material including PVA film, and the substrate part may be formed of a biodegradable material including kraft paper, so that the urine testing kit can be discharged into the toilet for sewage treatment after use.

### BRIEF DESCRIPTION OF DRAWINGS

FIG. 1 is a perspective view illustrating a urine testing kit of the present disclosure.

FIG. 2 is a perspective view illustrating a state of the urine testing kit of the present disclosure in which a cover part and a substrate part are separated.

FIG. 3 is a cross-sectional view taken along line A-A of FIG. 1.

FIG. 4 is a photograph showing reactivity between PVA and reagent papers.

FIG. 5 shows photographs showing discoloration of reagent papers in a urine testing kit exposed to moisture.

FIG. 6 shows photographs showing discoloration of reagent papers in a urine testing kit when spraying glucose 500 reagent.

### BEST MODE FOR CARRYING OUT THE INVENTION

A urine testing kit of the present disclosure includes a reagent paper which reacts with urine; a substrate part having a seating groove formed on its upper surface in which the reagent paper is seated; and a cover part which is coupled to the upper surface of the substrate part and covers an entrance of the seating groove, wherein a pore hole may be formed in the cover part through which the urine sprayed on an upper surface of the cover part is injected into the seating groove.

In the urine testing kit of the present disclosure, the pore holes may be formed at a density (number/cm²) of 4/cm² to 20/cm² in a position facing the entrance of the seating groove, and a diameter of the pore hole may be formed to be less than or equal to 1.0 mm.

In the urine testing kit of the present disclosure, a plurality of the seating grooves may be provided, and the pore holes may be formed for the respective seating grooves.

In the urine testing kit of the present disclosure, a plurality of the reagent papers may be provided, and each of the reagent papers may be placed in a different seating groove.

In the urine testing kit of the present disclosure, the reagent paper may be seated in close contact with a bottom surface of the seating groove, and a distance from a lower surface of the cover part to an upper surface of the reagent paper may be 1.0 mm or less.

In the urine testing kit of the present disclosure, a material of the substrate part may include one or more of kraft paper, sugarcane resin, paper, and corn starch.

In the urine testing kit of the present disclosure, a material of the cover part may be a hydrophilic material including one or more of Polyvinyl alcohol, Poly Latic Acid, Polycaprolactone, Sodium Carboxymethyl Cellulose, Polyhydroxyalkanoates (PHAs), Polybutylene adipate terephthalate (PBAT), Polycaprolactone (PCL), and Polybutylene succinate (PBS).

In the urine testing kit of the present disclosure, a plurality of the seating grooves may be provided, the substrate part may be provided in the form of a stick extending in a first direction, and the plurality of seating grooves may be aligned along the first direction.

In the urine testing kit of the present disclosure, the cover part may be formed of a transparent material.

### Modes for Carrying Out the Invention

Hereinafter, embodiments according to the present disclosure will be described in detail with reference to the accompanying drawings. In this process, the size or shape of the components shown in the drawings may be exaggerated for clarity and convenience of explanation. In addition, terms specifically defined in consideration of the configuration and operation of the present disclosure may vary according to the intentions or customs of users and operators. Definitions of these terms should be made based on the content throughout this specification.

In the description of the present disclosure, it should be noted that an orientation or positional relationship indicated by the terms such as “center”, “upper”, “lower”, “left”, “right”, “vertical”, “horizontal”, “inner side”, and “outer side” is based on an orientation or positional relationship shown in a drawing or an orientation or positional relationship that is placed when using the product of the present disclosure on a daily basis, and is merely for explanation and brief description of the present disclosure, and it does not suggest or imply that the displayed device or element must necessarily be configured or operated in a specified orientation and should not be construed as limiting the present disclosure.

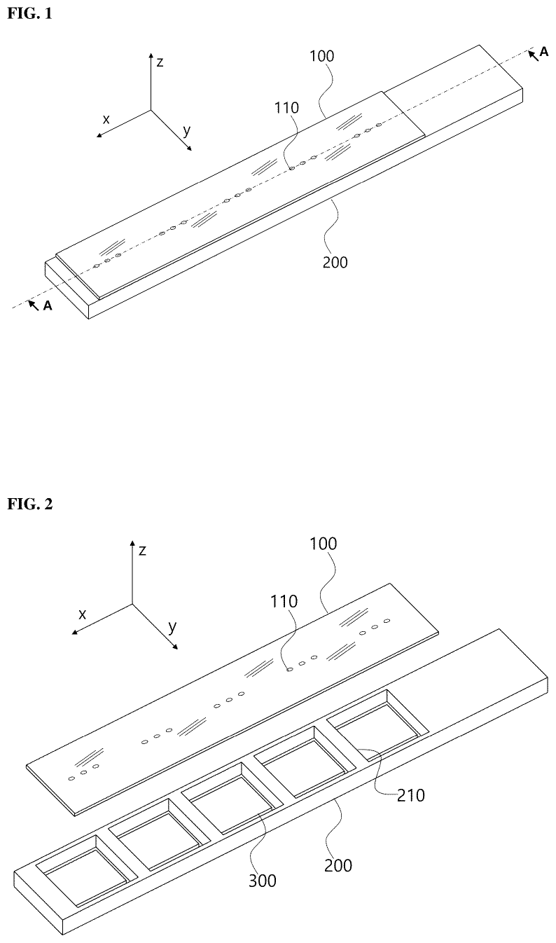

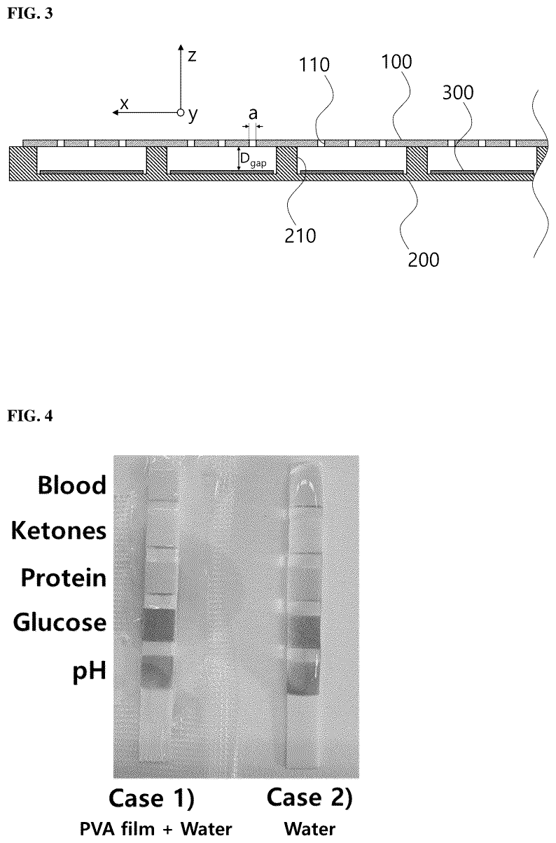

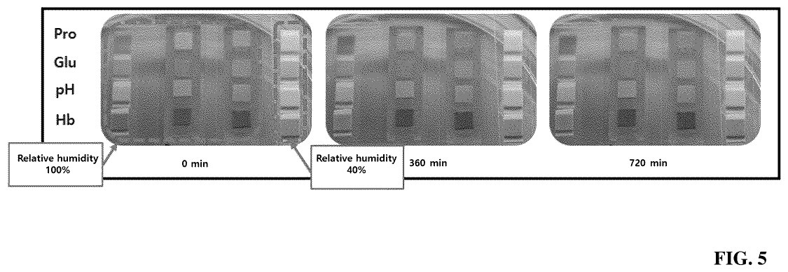

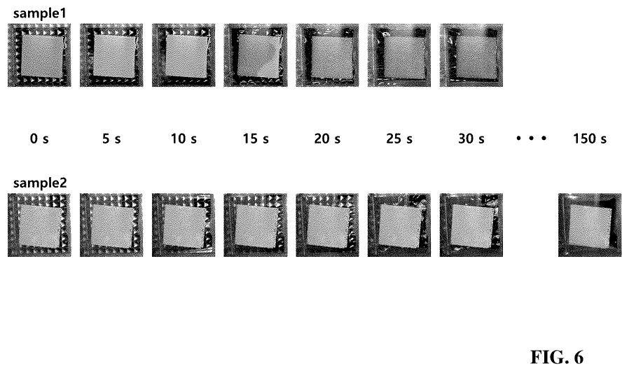

FIG. 1 is a perspective view illustrating a urine testing kit of the present disclosure. FIG. 2 is a perspective view illustrating a state of the urine testing kit of the present disclosure in which a cover part and a substrate part are separated. FIG. 3 is a cross-sectional view taken along line A-A of FIG. 1. FIG. 4 is a photograph showing reactivity between PVA and reagent papers. FIG. 5 shows photographs showing discoloration of reagent papers in a urine testing kit exposed to moisture. FIG. 6 shows photographs showing discoloration of reagent papers in a urine testing kit when spraying glucose 500 reagent.

Hereinafter, with reference to FIGS. 1 to 6, the urine testing kit of the present disclosure will be described in detail.

As shown in FIGS. 1 and 2, the urine testing kit of the present disclosure may include a reagent paper 300 that reacts with urine; a substrate part 200 having a seating groove 210 formed on its upper surface in which the reagent paper 300 is seated; and a cover part 100 which is coupled to the upper surface of the substrate part 200 and covers an entrance of the seating groove 210. A vertical direction may be the z-axis direction in FIGS. 1 to 3. Each of the substrate part 200 and the cover part 100 may be provided in the shape of a flat plate perpendicular to the z-axis, and the seating groove 210 may be formed on the upper surface of the substrate part 200. The cover part 100 may be coupled so that its lower surface faces the upper surface of the substrate part 200, thereby covering the entrance of the seating groove 210 to prevent external moisture from penetrating into the inner space of the seating groove 210.

In the urine testing kit of the present disclosure, a pore hole 110 may be formed in the cover part 100 through which the urine sprayed on an upper surface of the cover part 100 is injected into the seating groove 210.

As shown in FIG. 3, the seating groove 210 may be formed so that its bottom surface is a plane perpendicular to the vertical direction, and the reagent paper 300 may be fixed in close contact with the bottom surface of the seating groove 210. The depth of the seating groove 210 may be formed so that a gap D_{gap} between an upper surface of the reagent paper 300 and the lower surface of the cover part 100 is less than or equal to 10 mm.

TABLE 1

| | |
|---|---|
| D_{gap} | Number of pore holes |

| [mm] | 0 | 1 | 3 | 5 |
|---|---|---|---|---|
| 0 | X | ◯ | ◯ | ◯ |
| 0.2 | X | ◯ | ◯ | ◯ |
| 0.3 | X | ◯ | ◯ | ◯ |
| 0.4 | X | X | ◯ | ◯ |
| 0.5 | X | X | ◯ | ◯ |
| 0.6 | X | X | X | ◯ |
| 0.7 | X | X | X | ◯ |
| 0.9 | X | X | X | ◯ |
| 1.0 | X | X | X | ◯ |
| 1.2 | X | X | X | X |
| 1.5 | X | X | X | X |
| 1.7 | X | X | X | X |
| 2.0 | X | X | X | X |

Table 1 above shows experiment results after flowing 600 μl of Glucose 500 reagent with the urine testing kit tilted at about 20 degrees. Specifically, as the experiment results for 52 urine testing kits designed with different numbers of pore holes 110 and D_{gap}, the urine testing kit marked with ‘O’ indicates that the fluid reached the reagent paper 300 within 60 seconds, and the urine testing kit marked with ‘X’ indicates that the fluid did not reach the reagent paper 300 within 60 seconds. The pore hole 110 was manufactured with a diameter (a) of 0.5 mm.

Table 1 above shows that, when the number of pore holes 110 is three (3) and D_{gap} exceeds 0.5 mm, the fluid did not reach the reagent paper 300, and when the number of pore holes 110 is five (5) and D_{gap} exceeds 1.0 mm, the fluid did not reach the reagent paper 300. This is because the cover part 100 shrinks when it comes into contact with fluid. Specifically, after the fluid touches the cover part 100, it dissolves and the surface shrinks, and if D_{gap} is excessively distant, it may be difficult for the fluid to reach the internal reagent paper through the pore hole 100.

However, since the urine testing kit may become vulnerable to external humidity when D_{gap} is 0 mm, in other words, the reagent paper 300 and the cover part 100 are in close contact, it may be desirable for the reagent paper 300 and the cover part 100 to be spaced apart from each other at a certain distance. For example, D_{gap} may be 0.4 mm or greater. Therefore, D_{gap} may be greater than 0 mm and less than or equal to 1.0 mm, or 0.4 mm to 0.5 mm. For example, the depth of the seating groove 210 may be formed so that the gap D_{gap} between the upper surface of the reagent paper 300 and the lower surface of the cover part 100 is 0.5 mm.

The reagent paper 300 may be formed of a material that discolors by reacting with some components contained in urine. A plurality of the reagent papers 300 may be provided, and each reagent paper 300 may react with different components and become discolored. In other words, a plurality of types of reagent papers 300 may be provided.

A plurality of the seating grooves 210 may be provided. The seating grooves 210 may be provided to correspond to the number of the reagent papers 300, so that one reagent paper 300 may be seated in one seating groove 210. In other words, each of the reagent papers 300 may be placed in a different seating groove 210. In other words, in the urine testing kit of the present disclosure, since each of the plurality of reagent papers 300 is arranged in each of the plurality of seating grooves 210 provided as independent test spaces, it is possible to exclude a situation where contamination to other adjacent reagent papers 300 occurs and interferes with accurate diagnosis.

The substrate part 200 may be provided in the form of a stick extending in a first direction (x-axis direction), and the plurality of seating grooves 210 may be formed to be aligned along the first direction. Therefore, urine applied to an upper surface of the cover part 100 at the time of urine injection flows along the longitudinal direction (the first direction) of the substrate part 200, and may be dispersed and injected into each of the plurality of reagent papers 300.

The cover part 100 may be formed of a transparent material, and a user may identify the discoloration of the reagent paper 300 with naked eyes. In other words, the cover part 100 may be formed of a light-transmitting material that transmits light in the visible light range.

A material of the substrate part 200 may include one or more of kraft paper, sugarcane resin, paper (polypropylene coating), and corn starch (Polyvinyl alcohol, Poly Latic Acid, Polycaprolactone), and a material of the cover part 100 may be a hydrophilic material including one or more of corn starch (Polyvinyl alcohol, Poly Latic Acid, Polycaprolactone), cellulose (Sodium Carboxymethyl Cellulose), Polyhydroxyalkanoates (PHAs), Polybutylene adipate terephthalate (PBAT), Polycaprolactone (PCL), and Polybutylene succinate (PBS). In the urine testing kit of the present disclosure, the cover part 100 formed of a biodegradable hydrophilic material normally prevents moisture, but when urine comes into contact with it, fluid such as the urine may be quickly delivered to the reagent paper 300 inside the seating groove 210 through the pore hole 110.

Since general materials have hydrophobic properties, it is difficult for liquids to penetrate through small holes such as the pore holes 110 that simulate a stomata structure. However, PVA, which has hydrophilic properties, overcomes these disadvantages and allows urine to easily seep into the small pore holes 110 of the stomata structure. In addition, while conventional sticks made of hydrophobic materials do not spread liquid uniformly, urine may be spread uniformly throughout the reagent paper 300 through hydrophilicity.

In the urine testing kit of the present disclosure, the cover part 100 with the pore hole 110 may function as a fluid inlet passage. When conventional open-type materials are used or general materials are used, there may be difficulty in delivering flowing fluid to the reagent paper. However, the urine testing kit of the present disclosure utilizes hydrophilic materials at room temperature, so that the fluid may easily flow into the pore hole 110 and the fluid may be stably induced to the reagent paper 300.

The urine testing kit of the present disclosure protects the reagent paper 300 from humidity normally by providing a structure of the cover part 100 formed of the hydrophilic material with the pore hole 110, but when a liquid such as urine comes into contact with it, the urine may be delivered quickly and fast to the reagent paper 300. In other words, it usually has moisture resistance, but when it comes into contact with the liquid, it quickly accepts the liquid into the seating groove 210.

The protection of the reagent paper 300 is the ability to be performed by a cover made of any material, but it is difficult to deliver the liquid to the reagent paper. To this end, in the present disclosure, by forming the pore hole 110 in the cover part 100 as a structure that simulates a stoma, it may be used as a passage through which the fluid can be delivered to the reagent paper. However, general materials have a hydrophobic state at room temperature. In other words, liquid that touches a surface with hydrophobic properties does not spread to the material and clumps together, so it is very difficult to pass through small holes. This becomes even more difficult when it is a flowing fluid. Therefore, in the present disclosure, a PVA material with hydrophilic properties even at room temperature was selected. PVA materials are still being used as eco-friendly materials in many places to replace vinyl (PVC) materials. Unlike other materials, a cover made of hydrophilic PVA has the property of spreading fluids along the PVA surface when it comes into contact with liquids. Therefore, the fluids that spread in this way have the property of being able to easily enter the small pore holes that simulate stomata, and through this, the fluid (urine) that touches the cover part 100 may be quickly and accurately delivered to the reagent paper inside the pore hole 110.

In addition, since PVA, which is the material of the cover part 100, does not chemically react with the reagent paper 300 used in the urine test strip, it is possible to prevent the material of the cover part 100 from deteriorating urine test results. In FIG. 4, Case 1) is to check reactivity between PVA and the reagent paper 300 by covering a PVA film and applying water thereon, and Case 2) is to compare color changes of the reagent paper with case 1 by applying water only on the urine reagent paper 300 without PVA.

For a total of 5 types of reagent papers 300, it was found that they were not reactive with PVA, and in the case of Case 1), biodegradability of capable of sufficiently dissolving the PVA film with water alone was found.

Further, in the urine testing kit of the present disclosure, the substrate part 200 is formed of a biodegradable paper material such as kraft paper instead of a plastic test stick used in existing test strips, so it is environmentally friendly and may be disposed of as sewage into the toilet immediately after use.

Kraft paper is a raw material used for paper straws in cafes in recent years and has a strength similar to that of the plastic material used in test stick, and has the property of being able to dissociate in water and maintain its shape at the same time during urine test measurement time.

In the urine testing kit of the present disclosure, the cover part 100 may be provided in a shape that mimics the stomata structure of a plant. In plants, stomata in plants act like the plant's lungs and create a passageway where the outside air meets cells inside the leaf, and perform the function of maintaining constant internal humidity without being affected by external humidity.

The pore hole 110 may be formed in a position facing the entrance of the seating groove 210, a plurality of the seating grooves 210 may be provided, and each pore hole 110 may be formed for each of the seating grooves 210.

The pore hole 110 may protect the reagent paper 300 from moisture existing in a space (e.g., a bathroom, etc.) where the urine testing kit of the present disclosure is stored and may be formed so that, when urine is sprayed on the upper surface of the cover part 110, the urine spreads to the reagent paper 300 within several to tens of seconds.

The diameter (a) of the pore hole 110 may be formed to be 1.0 mm or less. For example, the diameter (a) of the pore hole 110 may be formed to be 0.5 mm, 0.65 mm, or 0.75 mm. By forming the pore hole 110 as described above, the reagent paper 300 may be maintained for a long time without discoloration even if it is left for a long time in the bathroom which is a high-humidity environment where the urine testing strip is mainly used.

TABLE 2

| Size of pore hole[mm] | 0.1 | 0.3 | 0.5 | 0.7 | 1.0 | 1.5 |
|---|---|---|---|---|---|---|
| Delivery time[s] | X | X | 10 | 6 | 4 | — |

Table 2 above shows experiment results after flowing 600 μl of Glucose 500 reagent with the urine testing kit tilted at about 20 degrees. Specifically, this shows results of an experiment in which six (6) urine testing kits formed with three (3) pore holes 110 and Dgap of 0.5 mm were prepared, and the diameter (a) of the pore holes 110 in each urine testing kit was set to 0.1 mm, 0.3 mm, 0.5 mm, 0.7 mm, 1.0 mmm, and 1.5 mm, respectively. When the size of the pore hole 110 was 0.1 mm or 0.3 mm, the size was too small to allow fluid to flow inside, making delivery of the fluid impossible, and when the size of the pore hole 110 was greater than 1.0 mm, the pore hole 110 became a passage through which the fluid could pass directly, so the meaning of the pore hole 110 disappeared and the reagent paper 300 could no longer be protected. Therefore, the diameter (a) of the pore hole 110 may be preferably 0.3 mm to 1.0 mm, and for example, the diameter (a) of the pore hole 110 may be 0.5 mm.

FIG. 5 shows photographs showing discoloration of the reagent papers 300 exposed to moisture. FIG. 5 may have been taken over time after exposing four (4) urine test kits to moisture. Each urine testing kit includes Pro, Glu, pH, and Hb as the reagent papers 300. In FIG. 5, three on the left of the four urine testing kits were exposed to 100% relative humidity, and one on the right was exposed to 40% relative humidity. Of the four urine testing kits, the middle two are the urine testing kits of the present invention with a cover part 100, and the rightmost and leftmost two are urine testing kits without a cover part 100.

Among the two urine testing kits in the middle, the left one has the pore hole 110 with a radius of 750 μm, and the right one has the pore hole 110 with a radius of 650 μm. As shown in FIG. 5, the reagent paper Pro and the reagent paper Hb change sensitively to water, and especially the reagent paper Pro is so sensitive that its color changes during setting, so a color change can be already seen in the 0-minute image.

As can be seen from the reagent paper Pro in FIG. 5, when the cover part 100 was not applied, the color changed to dark after being exposed for only 5 minutes at a relative humidity of 100%, and when the cover part 100 was applied, there was only a slight discoloration of the reagent paper.

As can be seen from the reagent paper Hb in FIG. 5, it gradually discolored orange over time for 360 minutes and 720 minutes when the cover part 100 was not applied, but it was seen that the color of the reagent paper was almost unchanged from the initial when the cover part 100 was applied.

In other words, even in the test in an extreme environment where the relative humidity in the bathroom is maintained 100% with the cover part 100 that simulates the stomata in the urine testing kit of the present disclosure, as a result of comparing changes in the reagent paper depending on the presence or absence of the cover part 100 for about 48 hours, it was found that there was a significant difference.

A plurality of the pore holes 110 may be provided, and the pore holes 110 may be arranged at a density (number/cm²) of 4/cm² to 20/cm² for each entrance of one seating groove 210. When the density of the pore holes 110 is 4/cm² or less, it is difficult for fluid to penetrate unless the reagent paper 300 is in contact, and when the density increases to 20/cm², humidity exchange occurs more actively within the 5 mm×5 mm size, making it difficult to preserve the reagent paper 300. As described above, the size of the pore hole 110 may converge to a certain value to prevent moisture from penetrating into the seating groove 210. Therefore, it may be necessary to secure an appropriate amount of the pore holes 100 to deliver urine sprayed on the upper surface of the cover part 100 to the inside of the seating groove 210 within a few to tens of seconds during which a person sprays the urine. For example, the reagent paper 300 is provided in a size of 5 mm×5 mm, and an amount of urine that can sufficiently wet the reagent paper 300 for a period of several to tens of seconds needs to pass through the cover unit 100. In consideration of the above, the pore holes 110 may be arranged with the density of 4/cm² to 20/cm² for each entrance of the seating groove 210.

FIG. 6 shows photographs showing results of sufficiently exposing reagent to a film by flowing 600 μl of Glucose 500 reagent with the urine testing kit tilted at about 20 degrees. In FIG. 6, sample 1 is one in which the pore hole 110 is formed in the cover part 100, and sample 2 is one in which no pore hole 110 is not formed in the cover part 100. In sample 1, a reaction occurred on the reagent paper six (6) seconds after the first contact with the reagent, but in sample 2, there was no change even after 2 minutes after the first contact.

Although embodiments according to the present disclosure have been described above, they are merely illustrative, and those skilled in the art will understand that various modifications and embodiments of an equivalent scope are possible therefrom. Accordingly, the true scope of technical protection of the present disclosure should be determined by the appended claims.

### EXPLANATION OF SYMBOLS

| | |
|---|---|
| 100 . . . Cover part | 110 . . . Pore hole |
| 200 . . . Substrate part | 210 . . . Seating groove |
| 300 . . . Reagent paper | |

### INDUSTRIAL APPLICABILITY

A urine testing kit of the present disclosure includes a cover part formed by a pore simulated cover structure, so that it is possible to minimize the effect of humidity on the reagent paper and increase the uniformity of reagent paper reaction.

In the urine testing kit of the present disclosure, each of a plurality of reagent papers is located in a space divided from each other, thereby allowing interference in results between the plurality of reagent papers to be eliminated.

In the urine testing kit of the present disclosure, a material of the cover part may be a hydrophilic and biodegradable material including PVA film, and the substrate part may be formed of a biodegradable material including kraft paper, so that the urine testing kit can be discharged into the toilet for sewage treatment after use.

1. A urine testing kit comprising:
a reagent paper which reacts with urine;
a substrate part having a seating groove formed on its upper surface in which the reagent paper is seated; and
a cover part which is coupled to the upper surface of the substrate part and covers an entrance of the seating groove,
wherein a pore hole is formed in the cover part through which the urine sprayed on an upper surface of the cover part is injected into the seating groove.

2. The urine testing kit of claim 1, wherein the pore holes are formed at a density (number/cm²) of 4/cm² to 20/cm² in a position facing the entrance of the seating groove, and a diameter of the pore hole is formed to be less than or equal to 1.0 mm.

3. The urine testing kit of claim 2, wherein a plurality of the seating grooves are provided, and
the pore holes are formed for the respective seating grooves.

4. The urine testing kit of claim 3, wherein a plurality of the reagent papers are provided, and
each of the reagent papers is placed in a different seating groove.

5. The urine testing kit of claim 1, wherein the reagent paper is seated in close contact with a bottom surface of the seating groove, and
a distance from a lower surface of the cover part to an upper surface of the reagent paper is 1.0 mm or less.

6. The urine testing kit of claim 1, wherein a material of the substrate part comprises one or more of kraft paper, sugarcane resin, paper, and corn starch.

7. The urine testing kit of claim 1, wherein a material of the cover part is a hydrophilic material comprising one or more of Polyvinyl alcohol, Poly Latic Acid, Polycaprolactone, Sodium Carboxymethyl Cellulose, Polyhydroxyalkanoates (PHAs), Polybutylene adipate terephthalate (PBAT), Polycaprolactone (PCL), and Polybutylene succinate (PBS).

8. The urine testing kit of claim 1, wherein a plurality of the seating grooves are provided,
the substrate part is provided in the form of a stick extending in a first direction, and
the plurality of seating grooves are aligned along the first direction.

9. The urine testing kit of claim 1, wherein the cover part is formed of a transparent material.
